(19) United States
(12) Patent Application Publication
(10) Pub. No.: US 2025/0172536 A1
(43) Pub. Date: May 29, 2025

(54) DEVICES, SYSTEMS, AND METHODS FOR MONITORING SOIL PROPERTIES

(71) Applicant: Smart Rain Systems, LLC, Bountiful, UT (US)

(72) Inventor: Rudy Lars Larsen, Bountiful, UT (US)

(21) Appl. No.: 18/962,773

(22) Filed: Nov. 27, 2024

Related U.S. Application Data
(60) Provisional application No. 63/603,517, filed on Nov. 28, 2023.

(51) Int. Cl. G01N 33/24

(52) U.S. Cl. CPC G01N 33/246

(57) ABSTRACT

A soil property sensor may include a housing having an upper end and a lower end and an opening. A soil property sensor may include a plurality of sensors located in the housing, the plurality of sensors including: a first soil property sensor located at the upper end of the housing; and a second soil property sensor located at the lower end of the housing. A soil property sensor may include a power source located in the housing, the power source providing power to the plurality of sensors. A soil property sensor may include a wireless communication system. A soil property sensor may include a sensor cover at the opening, the sensor cover forming a waterproof seal into the opening.

### CROSS-REFERENCE TO RELATED APPLICATIONS

This application claims priority to and benefit of U.S. Provisional Patent Application No. 63/603,517, filed Nov. 28, 2023, the disclosure of which is incorporated herein by reference in its entirety.

### BACKGROUND

Irrigation systems provide water to plants from a main water line, or water main. The water main has a capacity, which is typically measured in terms of a volumetric flow rate, such as gallons per minute (GPM). The water main capacity may be measured using other hydraulic parameters, such as pressure. Some irrigation systems may use one or more irrigation devices, such as a sprinkler head. These may help to deliver the irrigation water over an area. In some situations, the watering area to be irrigated (or watered) may utilize more water than is available from the water main. To water the entire watering area, an irrigation operator may separate this area into two or more zones. The irrigation devices in the zones may be connected to the water main through a valve. The valve may be opened and closed using an irrigation controller. In some situations, the irrigation devices may include soil property sensors to measure one or more soil properties.

### BRIEF SUMMARY

In some aspects, the techniques described herein relate to a soil property sensor. The soil property sensor includes a housing having an upper end and a lower end and an opening. The soil property sensor includes a plurality of sensors located in the housing. The sensors include: a first soil property sensor located at the upper end of the housing and a second soil property sensor located at the lower end of the housing. The soil property sensor includes a power source located in the housing. The power source provides power to the plurality of sensors. The soil property sensor includes a wireless communication system. The soil property sensor includes a sensor cover at the opening. The sensor cover forms a waterproof seal into the opening.

In some aspects, the techniques described herein relate to a method for soil monitoring in an irrigation system. A soil monitoring system receives or measures a first soil property measurement from a first sensor of a soil property sensor inserted in soil. The first sensor is located at a first sensor depth. The soil monitoring system receives or measures a second soil property measurement from a second sensor of the soil property sensor. The second sensor located at a second sensor depth. Based on the first soil property measurement and the second soil property measurement, the soul monitoring system generates a soil property gradient of said soil at the soil property sensor.

This summary is provided to introduce a selection of concepts that are further described below in the detailed description. This summary is not intended to identify key or essential features of the claimed subject matter, nor is it intended to be used as an aid in limiting the scope of the claimed subject matter. Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of a cloud-based irrigation system, according to at least one embodiment of the present disclosure.

FIG. 2 is a perspective view of a representation of a soil property sensor, according to at least one embodiment of the present disclosure.

FIG. 3 is a schematic representation of a soil monitoring system, according to at least one embodiment of the present disclosure.

FIG. 4 is a representation of a soil monitoring system with a soil property sensor installed in the ground adjacent to or proximate to a plant, according to at least one embodiment of the present disclosure.

FIG. 5 is a representation of a soil monitoring system, according to at least one embodiment of the present disclosure.

FIG. 6 is a flowchart of a method for soil monitoring, according to at least one embodiment of the present disclosure.

FIG. 7 illustrates certain components that may be included within a computer system.

### DETAILED DESCRIPTION

This disclosure generally relates to devices, systems, and methods for soil sensing. A soil property sensor may include multiple sensors. The sensors for the soil property sensor may measure and/or provide measurements for an inference for soil properties, such as soil moisture content, soil moisture gradient, soil temperature, soil pH, soil chemical properties, soil nutrient content, any other soil property, rates of change thereof, and combinations thereof. The soil property sensor may wirelessly transmit the measurements and/or soil properties to an irrigation controller. The irrigation controller may, based on the soil properties, adjust one or more watering programs. In this manner, the soil property sensor may improve the watering efficiency and/or reduce water usage of the irrigation system.

In accordance with at least one embodiment of the present disclosure, the soil property sensor may include two moisture sensors. A first moisture sensor may be located at an upper portion of the soil property sensor and a second moisture sensor may be located at a lower portion of the soil property sensor. The change in moisture levels between the first moisture sensor and the second moisture sensor may allow a soil monitoring system to determine the moisture gradient at the soil sensor. Plants often absorb water and/or nutrients within the top 12-18 inches of the soil, although other plants may absorb water and/or nutrients at greater and/or lower depths. In this manner, identifying the moisture gradient may help to identify how much moisture is available for the plant to absorb at various depths below the soil.

In some embodiments, the soil property sensor may include any other sensor. For example, the soil property sensor may include a pH sensor. The pH sensor may determine the pH of the soil. The pH of the soil may provide an indication of the chemical properties of a soil. Indeed, some plants more favorably grow in different pH soils. The plants and/or fertilizers planted in the soil may change the pH of the soil. Monitoring the pH using the pH sensor on the soil property sensor may allow the irrigation operator to make adjustments to the plants and/or fertilizers. This may help to improve the plant quality and/or plant health of plants in the irrigation system.

In some embodiments, the soil property sensor may collect time-series measurements. This may allow the irrigation operator to determine rates of change of the soil properties. As a specific, non-limiting example, the soil monitoring system may collect time-series measurements of soil moisture at the upper and lower moisture sensors. Using the time-series measurements, the soil monitoring system may determine the rate of change in moisture levels in the soil. In some embodiments, using the time-series measurements, the soil monitoring system may determine the rate of change of the moisture gradient. Using the rates of change of the moisture levels and/or the moisture gradient, the irrigation operator may make adjust the watering program. This may help to improve the plant quality and/or plant health of the plants in the irrigation system.

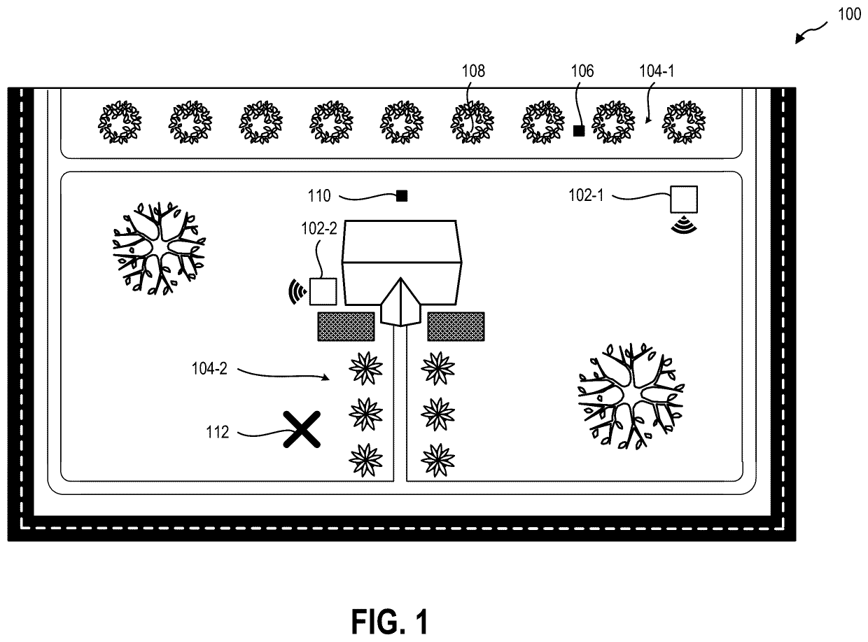

FIG. 1 is a representation of a cloud-based irrigation system 100, according to at least one embodiment of the present disclosure. The cloud-based irrigation system 100 includes a plurality of irrigation controllers (collectively 102). The irrigation controllers 102 may control the watering for a watering area. The watering area may include one or more watering zones (collectively 104). Each watering zone may include one or more irrigation devices 106. The irrigation devices 106 may provide water to one or more plants 108. The irrigation devices 106 may include any type of irrigation device, such as a spray head, a rotary head, a drip irrigation device, any other type of irrigation device, and combinations thereof. In some embodiments, an irrigation device 106 may water a single plant 108. In some embodiments, an irrigation device 106 may provide water to multiple plants 108. The plants 108 may include any type of plant, such as grass, shrubs, flowers, trees, garden plants, crops, any other type of plant, and combinations thereof.

Different irrigation controllers 102 may provide water to different irrigation zones 104. For example, a first irrigation controller 102-1 may control the irrigation to a first zone 104-1 and a second irrigation controller 102-2 may control the irrigation to a second zone 104-2. While two irrigation controllers 102 are shown each controlling a single zone 104, it should be understood that the watering area may include any number of irrigation controllers 102 watering any number of zones 104. For example, a single irrigation controller 102 may control 48 zones or any number of zones.

The irrigation controllers 102 may include any type of irrigation controller. For example, the irrigation controllers 102 may include a single-wire irrigation controller, with multiple valves or other irrigation devices connected to a single common wire. In some examples, the irrigation controllers 102 may include a “two-wire controller.” A two-wire controller may connect multiple valves or other irrigation devices in series. A two-wire controller may allow for an increased number of connected valves. In some embodiments, the irrigation controllers 102 may include a wireless irrigation controller. For example, the irrigation controllers 102 may wirelessly connect to the valves and other connected irrigation devices. In some embodiments, the irrigation controllers 102 may include any type of irrigation controller connected to one or more valves in any way, including wired connections having any configuration, wireless connections, and combinations thereof. In some embodiments, the cloud-based irrigation system 100 may include multiple types of irrigation controllers 102.

The irrigation controllers 102 may include local irrigation programs for their associated zones 104. In some embodiments, an irrigation program may include a time of day to water for a particular zone 104 and/or set of zones 104. In some embodiments, an irrigation program may include a duration to water for a particular zone 104 and/or set of zones 104. In some embodiments, an irrigation program may include times during which to collect irrigation conditions from one or more sensors 110, as discussed herein. In some embodiments, the irrigation program may be a local irrigation program associated with the particular irrigation controller 102. In some embodiments, a local irrigation program may include controls for any other operation or combinations of operations performed by the irrigation controller 102.

As may be seen, the irrigation controllers 102 may be physically located within one of the zones 104 they control and/or proximate one or more of the zones 104 they control. For example, the first irrigation controller 102-1 is shown as proximate to the first zone 104-1. The second irrigation controller 102-2 is shown as proximate to the second zone 104-2. The irrigation controllers 102 may be located within or proximate the single zone 104 to reduce the wiring length and/or the piping to the valves and/or irrigation devices 106 of the respective zones.

The cloud-based irrigation system 100 may include one or more sensors 110. The sensors 110 may detect irrigation conditions of the cloud-based irrigation system 100. For example, the sensors 110 may detect irrigation conditions of plants and/or soil in the watering area. For example, the sensors 110 may detect soil moisture, soil pH, soil chemistry, and other irrigation conditions of the plants and/or soil in the watering area. In some examples, the sensors 110 may detect flow rate information for one or more of the zones 104 and/or irrigation devices 106. In some examples, the sensors 110 may detect pressure information for one or more of the zones 104 and/or irrigation devices 106. In some examples, the sensors 110 may collect weather information. For example, the sensors 110 may detect temperature, humidity, rainfall, wind speed, wind direction, barometric pressure, any other weather information, and combinations thereof. In some embodiments, the sensors 110 may collect photographic information of all or a portion of the watering area. For example, the sensors 110 may collect photos of a patch of grass or plants. Plants may change colors based on over-watering, under-watering, nutrient levels, the presence of pests, the presence of weeds, temperature, any other condition, and combinations thereof.

In accordance with at least one embodiment of the present disclosure, the sensors 110 may include one or more soil property sensors 112. The soil property sensor 112 may be inserted into the soil in one or more of the irrigation zones 104. When inserted into the ground, the soil property sensor may measure one or more soil properties using one or more sensors. For example, the one or more soil property sensors 112 may include one or more of a moisture sensor, a temperature sensor, a pH sensor, a soil chemistry sensor, any other soil sensor, and combinations thereof.

In some embodiments, the soil property sensor 112 includes a plurality of the same type of sensor. For example, and as discussed in further detail herein, the soil property sensor 112 may include two moisture sensors. The moisture sensors may be located at different depths along the length of the soil property sensor 112. A soil monitoring system may receive the soil measurements and determine a moisture gradient along the depth of the soil property sensor 112. The moisture gradient may be a representation of the change in soil moisture along the length of the soil property sensor 112.

Using the soil moisture gradient, the soil monitoring system may analyze the irrigation program. For example, the soil monitoring system may analyze whether a particular irrigation program provides sufficient water to moisten the soil to a target soil moisture level at a particular depth. This may help to ensure that the entire moisture and/or nutrient absorbing portions of the roots of a particular plant have access to irrigation water. In this manner, generating the moisture gradient may allow the irrigation operator to determine the effectiveness of the irrigation program. In some embodiments, the irrigation operator and/or the cloud-based irrigation system 100 may change or adjust the irrigation program based on the moisture gradient. This may help to improve the plant quality and/or plant health.

In some embodiments, the soil monitoring system may monitor other soil properties. For example, the soil monitoring system may monitor soil chemistry. In some examples, the soil monitoring system may monitor pH. If the pH of the soil is different than a target pH, then the soil monitoring system may provide an irrigation and/or landscaping recommendation to adjust the pH of the soil. In some examples, the recommendation may include a fertilizer recommendation, a plant recommendation (e.g., a type of plant that may adjust the pH of the soil), a microorganism recommendation (e.g., a microorganism added to the soil that may adjust the pH of the soil), any other irrigation and/or landscaping recommendation, and combinations thereof.

In some embodiments, the soil monitoring system monitors temperature. In some embodiments, the soil monitoring system may identify when the temperature has reached above and/or below a threshold temperature. For example, if the soil temperature has, such as in the late winter and/or early spring, reached above a planting temperature (e.g., a temperature above which a particular plant may grow and/or thrive), the soil monitoring system may prepare a recommendation to plant at or around the location of the soil property sensor 112. In some examples, if the soil temperature has dropped below a threshold temperature, then the soil monitoring system may prepare a recommendation to winterize a set of plants (e.g., dig up annuals, cut back multi-annuals/perennials, dead-head plants). In some examples, if the soil temperature has exceeded a high threshold temperature, then the soil monitoring system may prepare a recommendation to increase the watering amount and/or time and/or to change the plants planted at or adjacent to the soil property sensor 112 to more heat-tolerant plants.

In some embodiments, the soil monitoring system may us a combination of measured soil properties to prepare the recommendation. For example, the soil monitoring system may use a combination of soil moisture gradient and soil temperature to determine the frequency and/or amount of irrigation to perform. In some examples, the soil monitoring system may use a combination of soil moisture gradient and pH, pH and temperature, or soil moisture gradient, pH, and temperature to determine a type of plant or plants that may thrive in the soil environment.

In some embodiments, the sensors 110, including soil property sensor 112, may be in communication with the irrigation controllers 102. For example, one or more of the sensors 110 may be located within a zone associated with a particular irrigation controller 102, and the sensors 110 may be in communication (e.g., wired or wireless communication) with the irrigation controller 102. Put another way, the sensors 110 may communicate (e.g., wired or wirelessly) with the irrigation controllers 102. In some embodiments, the sensors 110 may receive power from the associate irrigation controller 102.

In accordance with at least one embodiment of the present disclosure, the soil property sensor 112 may include a wireless transmitter. When the soil property sensor 112 measures one or more soil properties, the soil property sensor 112 may wirelessly transmit the measurements to the irrigation controller 102. The soil property sensor 112 may include an independent power source. This may allow the soil property sensor 112 to operate without wired power from the grid and/or the irrigation controller 102. In some embodiments, the wireless transmitter may include a low-power transmitter, such as a LoRa (long range) radio transmitter.

In accordance with at least one embodiment of the present disclosure, the irrigation controllers 102 may be in communication with the internet. Put another way, the irrigation controllers 102 may include a communication device that connects the irrigation controllers 102 to the internet. In some embodiments, the irrigation controllers 102 may have two-way communication with the internet. In some embodiments, the irrigation controllers 102 may be tethered to a cloud-based irrigation controller. For example, the irrigation controllers 102 may be tethered to the cloud-based irrigation controller over the internet. The cloud-based irrigation controller may receive irrigation information from the irrigation controllers 102 and provide irrigation instructions to the irrigation controllers 102.

While irrigation controllers 102 are described and illustrated as being in communication over the internet, it should be understood that the irrigation controllers may operate independent of the internet. For example, the irrigation controllers 102 may include local memory. The local memory may include the irrigation program for the local irrigation controller 102. The local irrigation controller 102 may implement the irrigation program from the local memory. This may help to reduce data usage and bandwidth requirements for the local irrigation controller 102. In some embodiments, if the local irrigation controller 102 is disconnected from the internet (for any reason), the local irrigation controller may still implement the local irrigation program. This may help to provide redundancy in the cloud-based irrigation system.

As discussed herein, the irrigation program may be stored on the local memory. In some embodiments, the local irrigation controller 102 may not be able to make changes to the irrigation program stored on the local memory. For example, an irrigation operator may not be able to adjust the irrigation program stored on the local memory. In some embodiments, the irrigation program may only be changed from an internet connection. In some embodiments, the irrigation program may be changed locally after receiving approval from the cloud-based irrigation controller to which the local irrigation controller 102 is tethered. This may help to prevent unintentional and/or unauthorized changes to the irrigation program.

In some embodiments, the irrigation controllers 102 may be constantly connected to the internet (except for unexpected internet outages). In some embodiments, the irrigation controllers 102 may periodically or episodically connect to the internet. For example, the irrigation controllers 102 may periodically connect to the internet every second, every 10 seconds, every 30 seconds, every minute, every 10 minutes, every 30 minutes, every hour, every 12 hours, every day, every week, every month, every season, every year, or any timeframe therebetween. In some embodiments, the irrigation controllers may episodically connect to the internet when an event occurs, such as when new equipment is installed, an alarm is triggered, a user instructs that irrigation controller, a certain amount of irrigation information has been collected, any other episode, and combinations thereof.

In some embodiments, the local memory may include a storage cache. The storage cache may store irrigation information collected by the local irrigation controller 102. In some embodiments, the local storage may store irrigation information until the irrigation information is uploaded to the cloud-based irrigation controller. For example, the local storage may store the irrigation information collected during an interruption to internet service, and may upload the irrigation information when internet service is restored. In some example the local storage may store the irrigation information between periodic or episodic internet connections, and may upload the stored irrigation information at the time of connection to the internet.

As discussed herein, the irrigation controllers 102 may provide the cloud-based irrigation controller with irrigation information. In some embodiments, the irrigation information may include irrigation conditions detected by the sensors 110, including the soil property sensor 112. In some embodiments, the irrigation information may include details of the operation of the irrigation controllers 102. For example, the irrigation information may include the time a zone is watered, the duration that a zone is watered, the flow rate associated with a zone, the pressure drop associated with a zone, any other information, and combinations thereof.

In some embodiments, the cloud-based irrigation controller may generate irrigation instructions and provide them to the tethered irrigation controllers 102. For example, the irrigation instructions may include a change to the irrigation program for one or more of the irrigation controllers 102. In some examples, the cloud-based irrigation controller may provide instructions regarding the time of day one or more zones 104 waters and/or the duration that one or more zones 104 waters.

The cloud-based irrigation controller may generate irrigation instructions using the irrigation information. For example, the cloud-based irrigation controller may generate the irrigation instructions based on the irrigation information received from the irrigation controllers 102. In some examples, the cloud-based irrigation controller may generate the irrigation instructions based on sensor readings from the sensors 110. The irrigation instructions may be at least partially based on the irrigation information. For example, the irrigation instructions may adjust a watering day, time of day and/or duration based on the irrigation information. In some examples, the irrigation instructions may adjust a watering day, time of day and/or duration based on irrigation conditions.

In some embodiments, the cloud-based irrigation controller may generate a master irrigation program. The master irrigation program may include watering days, times of day, and durations for the zones 104 in the watering area. For example, the master irrigation program may include watering days, times of day, and durations for each zone 104 associated with each irrigation controller 102 tethered to the cloud-based irrigation controller.

The master irrigation program may coordinate the watering between the various irrigation controllers 102 and zones 104. For example, the master irrigation program may coordinate the flow from the water main, improving use of the water supply during the watering window. In some embodiments, the master irrigation plan may be flexible and adaptable based on the changing irrigation conditions of the entire watering area. In this manner, the watering efficiency of the watering area may be improved, which may save money, water, and/or time.

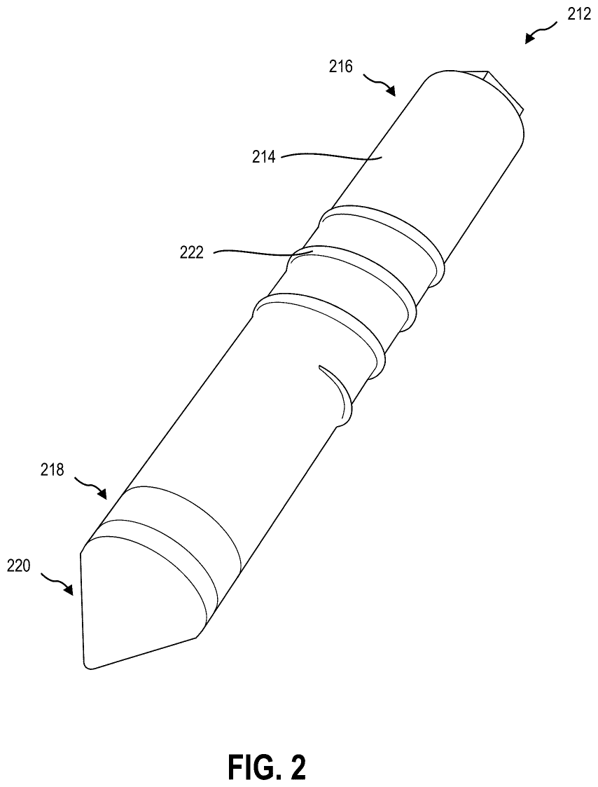

FIG. 2 is a perspective view of a representation of a soil property sensor 212, according to at least one embodiment of the present disclosure. The soil property sensor 212 shown includes a body 214, an upper end 216, and a lower end 218. The soil property sensor 212 is configured to be inserted into the ground. The lower end 218 may be inserted into the ground first and may be deeper underground than the upper end 216. In some embodiments, the upper end 216 is configured to be flush with the surface of the ground. In some embodiments, the upper end 216 is configured to be located underground. In some embodiments, the upper end 216 is configured to extend above the surface.

In some embodiments, the soil property sensor 212 may include one or more installation and/or retention aids. For example, the lower end 218 may include a pointed tip 220. The pointed tip 220 at the lower end 218 may take any shape, including conical, pyramidal, or other pointed shape. The pointed tip 220 at the lower end lower end 218 may aid in the installation of the soil property sensor 212 in the soil by helping push the soil away when driving the soil property sensor 212 into the soil. In some embodiments, the pointed tip 220 at the lower end 218 may be used to guide the soil property sensor 212 into a pilot hole or other pre-drilled or pre-dug hole in the soil.

While the embodiment shown in FIG. 2 is illustrated with the pointed tip 220, it should be understood that the soil property sensor 212 may include any tip, including a flat tip, a domed tip, a concave tip, any other shape tip, and combinations thereof. For example, the soil property sensor 212 may be installed by digging or drilling a hole in the soil, and the soil property sensor 212 placed or driven into the pre-drilled hole.

In the embodiment shown, the soil property sensor 212 may include retention features 222. The retention features 222 may include extensions from the outer surface of the body 214. The retention features 222 may help to engage the soil in which the soil property sensor 212 is installed and increase the force required to remove the soil property sensor 212. This may help to secure the soil property sensor 212 in the ground, thereby reducing the unintentional or inadvertent removal of the 212. In some embodiments, the retention features 222 may help with installation of the soil property sensor 212. For example, the retention features 222 may include spiral extensions from the outer surface of the body 214. When installed in the ground, the spiral extensions may, when the soil property sensor 212 is rotated, help to drive the soil property sensor 212 into the ground.

As discussed herein, the soil property sensor 212 may include soil sensors. The soil sensors may be located at any position on or in the soil property sensor 212. For example, the soil sensors may be located in an interior of the soil property sensor 212. Placing the soil sensors in the interior of the soil property sensor 212 may help to protect the soil sensors from the environment of the soil. This may help to extend the operational lifetime of the soil property sensor 212. In some embodiments, the soil sensors may be located on an outer surface of the body 214 and/or exposed to the soil when installed in the ground. This may help to increase the accuracy and/or representation of the measurements measured by the exposed soil sensors.

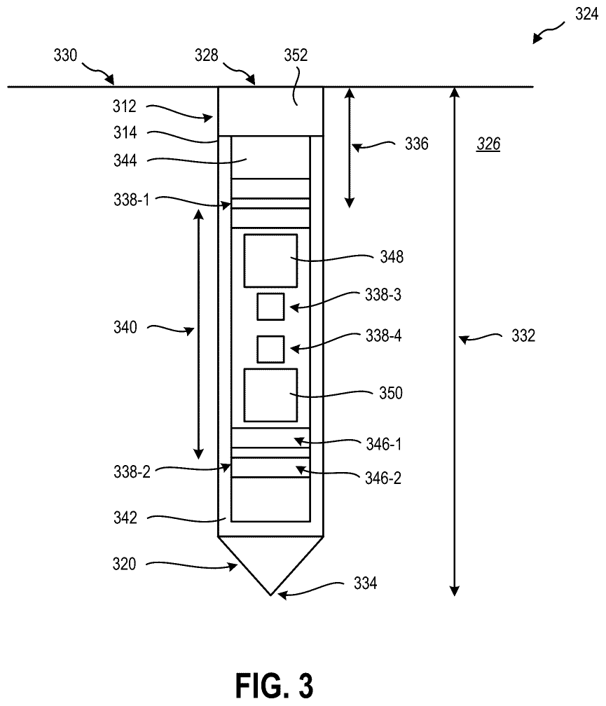

FIG. 3 is a schematic representation of a soil monitoring system 324 including a cut-away view of a soil property sensor 312 installed in the ground 326, according to at least one embodiment of the present disclosure. The soil property sensor 312 may be installed in the ground 326 such that an upper surface 328 of the soil property sensor 312 is flush with a surface 330 of the ground 326. This may help to prevent the soil property sensor 312 from interfering with landscaping activities, including mowing grass, weeding, planting plants, fertilizing, irrigation, installation of irrigation equipment, any other landscaping activities, and combinations thereof. In some embodiments, the upper surface 328 may be uncovered and exposed to the atmosphere. This may help to improve communication signals between the soil property sensor 312 and an irrigation controller.

While the soil property sensor 312 of FIG. 3 has been shown as installed with the upper surface 328 flush with the surface 330, it should be understood, that the upper surface 328 may extend above the surface 330. This may help to increase the visibility of the soil property sensor 312, allowing the irrigation operator to access the soil property sensor 312 for troubleshooting, repair, and/or replacement. In some examples, the upper surface 328 may be located at an elevation below the surface 330. In some examples, the upper surface 328 may be at least partially covered with soil, mulch, wood chips, plants, or other landscaping material.

The soil property sensor 312 has an instrument length 332. The instrument length 332 may be the length of the soil property sensor 312 from the upper surface 328 to the lower surface 334 (e.g., the bottom point of the pointed tip 320). In some embodiments, the instrument length 332 may be in a range having an upper value, a lower value, or upper and lower values including any of 3 in. (7.6 cm), 6 in. (15.2 cm), 9 in. (22.9 cm), 12 in. (30.5 cm), 15 in. (38.1 cm), 18 in. (45.7 cm), 21 in. (53.3 cm), 24 in. (61.0 cm), or any value therebetween. For example, the instrument length 332 may be greater than 3 in. (7.6 cm). In another example, the instrument length 332 may be less than 24 in. (61.0 cm). In yet other examples, the instrument length 332 may be any value in a range between 3 in. (7.6 cm) and 24 in. (61.0 cm). In some embodiments, it may be critical that the instrument length 332 is between 12 in. (30.5 cm) and 18 in. (45.7 cm) to measure soil properties where the roots may absorb water and/or nutrients.

As discussed herein, the soil property sensor 312 may be installed in the ground 326 with the upper surface 328 flush or approximately flush with the surface 330. This may result in the lower surface 334 being at an installation depth (e.g., the depth below the surface 330 that the lower surface 334 is located when installed in the ground 326) that is the same as or approximately the same as the instrument length 332. However, it should be understood that the installation depth may be different than the instrument length 332, including greater than (e.g., the upper surface 328 is located below the surface 330) or less than (e.g., the upper surface 328 is located above the surface 330) the instrument length 332.

As discussed herein, the soil property sensor 312 may include a plurality of sensors. The sensors may measure soil properties, such as moisture content, temperature, pH, and other soil properties. The sensors may be located at any point along the instrument length 332. When the upper surface 328 is installed flush with the surface 330, the location of the sensors along the instrument length 332 may be the same as or approximately the same as a sensor depth 336 below the surface 330. However, it should be understood that the sensor depth 336 may be different than the location of the sensor along the instrument length 332, based on the relative position of the upper surface 328 with respect to the surface 330.

In some embodiments, the sensor depth 336 may be in a range having an upper value, a lower value, or upper and lower values including any of 0.5 in. (1.3 cm), 1 in. (2.5 cm), 1.5 in. (3.8 cm), 3 in. (7.6 cm), 6 in. (15.2 cm), 9 in. (22.9 cm), 12 in. (30.5 cm), 15 in. (38.1 cm), 18 in. (45.7 cm), 21 in. (53.3 cm), 24 in. (61.0 cm), or any value therebetween. For example, the sensor depth 336 may be greater than 0.5 in. (1.3 cm). In another example, the sensor depth 336 may be less than 24 in. (61.0 cm). In yet other examples, the sensor depth 336 may be any value in a range between 0.5 in. (1.3 cm) and 24 in. (61.0 cm). In some embodiments, it may be critical that the sensor depth 336 is between 0.5 in. (1.3 cm) and 24 in. (61.0 cm) to measure soil properties where the roots may absorb water and/or nutrients. As may be understood, many plants absorb water and nutrients within the top 6 to 18 inches of the ground. Measuring soil properties within the top 6 to 18 inches may help to determine the properties of the soil in the location that has the highest impact on the plant.

In accordance with at least one embodiment of the present disclosure, the soil property sensor 312 includes a plurality of sensors (collectively 338), including a first sensor 338-1 located at a first sensor depth 336, a second sensor 338-2 located at a second sensor depth 336, a third sensor 338-3 located at a third sensor depth 336, and a fourth sensor 338-4, located at a fourth sensor depth 336.

The sensors 338 may be separated with a sensor separation 340. The sensor separation may be the distance between two sensors 338. In some embodiments, the sensor separation 340 may be in a range having an upper value, a lower value, or upper and lower values including any of 0.5 in. (1.3 cm), 1 in. (2.5 cm), 1.5 in. (3.8 cm), 3 in. (7.6 cm), 6 in. (15.2 cm), 9 in. (22.9 cm), 12 in. (30.5 cm), 15 in. (38.1 cm), 18 in. (45.7 cm), 21 in. (53.3 cm), or any value therebetween. For example, the sensor separation 340 may be greater than 0.5 in. (1.3 cm). In another example, the sensor separation 340 may be less than 21 in. (53.3 cm). In yet other examples, the sensor separation 340 may be any value in a range between 0.5 in. (1.3 cm) and 21 in. (53.3 cm). In some embodiments, it may be critical that the sensor separation 340 is between 3 in. (7.6 cm) and 21 in. (53.3 cm) to measure soil properties across the instrument length 332 and generate a representation of the soil properties at locations where the roots may absorb water and nutrients.

In some embodiments, two of the same type of sensors 338 that are located at different sensor depths 336 may measure different values of a soil property. This may be a result of soil conditions that may change based on the depth below the surface 330. In accordance with at least one embodiment of the present disclosure, using the differential soil property measurements, the soil monitoring system 324 may generate a property gradient of the soil property. The property gradient may be a representation of the change in the soil property across the depth into the ground 326.

Using the property gradient, the soil monitoring system 324 may identify soil properties at any depth. In some embodiments, the soil monitoring system 324 may identify soil properties at depths between the sensors 338 that collected the measurements used to generate the property gradient. In some embodiments, the soil monitoring system 324 may identify soil properties at depths above or below the depths of the relevant sensors 338. For example, the property gradient may be estimated as a function of soil property with respect to depth. The function may be any function, such as a linear function, a polynomial function of any order, a hyperbolic function, an exponential function, any other function, and combinations thereof. The soil monitoring system 324 may apply a target depth different from the sensor depth 336 to the soil property function to estimate or infer the soil property at the target depth. In this manner, by using multiple sensors 338 that measure the same property, the soil monitoring system 324 may determine soil properties at any depth along the instrument length 332. In some examples, the soil monitoring system 324 may determine soil properties at depths greater than the instrument length 332.

As discussed herein, the sensors 338 may be any type of sensor. In some embodiments, each of the sensors 338 are the same type of sensor. In some embodiments, each of the sensors 338 are different types of sensors. In some embodiments, the sensors 338 may include a combination of multiples of the same type of sensor and different sensors.

As a specific, non-limiting example, at least two of the sensors 338 may include moisture sensors that prepare or measure moisture measurements. For example, the first sensor 338-1 and the second sensor 338-2 may include moisture sensors. The first sensor 338-1 and the second sensor 338-2 may be separated by the sensor separation 340. The first sensor 338-1 may measure a first soil moisture content and the second sensor 338-2 may measure a second moisture content. The soil monitoring system 324 may generate a moisture gradient based on the first moisture content and the second moisture content. The moisture gradient may allow the irrigation operator to determine, infer, or estimate the moisture content at any depth. For example, the moisture gradient may allow the irrigation operator to determine, infer, or estimate the moisture content at any depth along the instrument length 332 and deeper than the instrument length 332.

Using the moisture gradient, the irrigation operator may prepare an irrigation recommendation. For example, the ground 326 may have a moisture content threshold or threshold range. At moisture contents outside of the moisture content threshold or threshold range, plants in the irrigation zone may not grow, or may have reduced plant quality or reduced plant health. The moisture gradient may indicate that the soil moisture content at a depth below the surface 330 is outside of the moisture content threshold. Based on this determination, the soil monitoring system 324 may prepare a recommendation to adjust an irrigation program to return the moisture content to within the threshold.

As a specific, non-limiting example, the moisture gradient may indicate that the moisture content is within the moisture content threshold or threshold range at a first depth below the surface 330 and outside of the moisture content threshold or threshold range at a second depth below the surface 330. Based on this determination, the soil monitoring system 324 may prepare a recommendation to adjust the irrigation program to return the moisture content to within the threshold. For example, the soil monitoring system 324 may prepare a recommendation to increase a watering time, thereby increasing the depth that the water soaks in the ground 326 to the determined depth.

The third sensor 338-3 may include a temperature sensor and the fourth sensor 338-4 may include a pH sensor. The soil property sensor 312 may collect temperature measurements with the third sensor 338-3 and pH measurements with the fourth sensor 338-4. Using the temperature and pH measurements, the soil monitoring system 324 may prepare further irrigation and/or landscaping recommendations. For example, the soil monitoring system 324 may prepare irrigation and/or landscaping recommendations for plant type and/or watering schedule that is tailored to the measurements in the ground 326.

While the example above is described with respect to moisture sensors and a moisture content gradient, it should be understood that the soil property gradient may be any type of soil property. For example, the first sensor 338-1 and the second sensor 338-2 may be temperature sensors, and the soil property gradient may be a temperature gradient. In some examples, the first sensor 338-1 and the second sensor 338-2 may be pH sensors, and the soil property gradient may be a pH gradient. In some embodiments, the first sensor 338-1 and the second sensor 338-2 may include multiple sensors at that sensor depth 336. This may allow for the determination of multiple soil property gradients across the same sensor separation 340.

In accordance with at least one embodiment of the present disclosure, the soil monitoring system 324 may collect time-series data from the sensors 338. Using the time-series data, the soil monitoring system 324 may collect rates of change of the soil properties and/or soil property gradients. This may allow the irrigation operator to identify changes in the soil properties over time. In some embodiments, the soil monitoring system 324 may prepare irrigation and/or landscaping recommendations based on the rates of change in soil properties.

As a specific non-limiting example, the rate of change of moisture gradient may provide the irrigation operator information regarding the rate of moisture depletion. In some examples, the rate of change of moisture gradient may provide the irrigation operator information regarding the rate of moisture depletion at various depths into the ground 326. This may allow the operator to identify and/or infer the amount of water absorbed by the plants, the amount of water that drains from the soil, the amount of water that evaporates out the surface 330 of the ground 326, any other rate of change information, and combinations thereof. Using the rate of change of the moisture gradient, the soil monitoring system 324 may generate a landscaping and/or irrigation recommendation. For example, the soil monitoring system 324 may generate a recommendation to increase the amount and/or frequency of waterings. In some examples, the soil monitoring system 324 may generate a recommendation to change the plant type. In some examples, the soil monitoring system 324 may generate a recommendation to change the soil type.

The soil monitoring system 324 may collect multiple rates of change of soil properties. For example, the soil monitoring system 324 may collect rates of change for any combination of moisture contents/gradients, temperature value/gradient, pH value/gradient, any other soil property, and combinations thereof. Using the combination of rates of change of a combination of soil properties, the soil monitoring system 324 may prepare an irrigation and/or landscaping recommendation.

In some embodiments, the soil property sensor 312 may include an outer sheath 342. An inner housing 344 may be inserted into the outer sheath 342. The inner housing 344 may include the sensors 338. For example, the inner housing 344 may include a tube inserted into a hollow bore in the outer sheath 342. The sensors 338 may be secured to the inner housing 344. In some embodiments, one or more of the sensors 338 are located in an interior of the inner housing 344. In some embodiments, one or more of the sensors 338 are secured to an outer surface of the inner housing 344.

As a specific, non-limiting example, a moisture sensor may be secured to an outer surface of the inner housing 344. As a specific, non-limiting example, the moisture sensor may include a capacitive moisture sensor. The capacitive moisture sensor may include a transmitter 346-1 and a receiver 346-2. The transmitter 346-1 may transmit an electromagnetic signal. The electromagnetic signal may pass into the ground 326, and the receiver 346-2 may receive the signal. The transmitter 346-1 and the receiver 346-2 may be connected to a capacitor, and the capacitance of the capacitor may be representative of the moisture content of the soil.

In the embodiment shown, the transmitter 346-1 and the receiver 346-2 are shown as wire or metal sheet bands around the outer surface of the inner housing 344. This may facilitate accurate measurements that are representative of a 360° space around the soil property sensor 312. In some embodiments, the transmitter 346-1 and the receiver 346-2 may be any type of transmitter and receiver, including exposed wire ends, probes, loop antennas, any other type of transmitter or receiver, and combinations thereof. In the embodiment shown, the transmitter 346-1 and the receiver 346-2 may not be in contact with the ground 326. This may help to increase the operating lifetime of the soil property sensor 312. But in some embodiments, the transmitter 346-1 and the receiver 346-2 may be in contact with the ground 326. This may help to increase the sensitivity of the moisture sensor. While a capacitive-type moisture sensor has been described herein, it should be understood that any type of moisture sensor may be used, such as a resistive, a time-domain reflectometry (TDR) sensor, any other moisture sensor, and combinations thereof.

The soil property sensor 312 may include a wireless communication system 348. The wireless communication system 348 may provide wireless communication with other elements of an irrigation system. For example, the wireless communication system 348 may provide wireless communication with wireless irrigation controllers, a cloud-based irrigation controller, other soil property sensors, wireless valves, wireless sensors, any other wireless irrigation element, and combinations thereof. The wireless communication system 348 may include a transmitter and/or a receiver. The wireless communication system 348 may include any type of wireless communication system. For example, the wireless communication system 348 may include a cellular communication system, a Wi-Fi communication system, Bluetooth, radio communication system, a LoRa communication system, any other wireless communication system, and combinations thereof. In some embodiments, the wireless communication system 348 may be a low-power communication system to reduce the drain on the power source.

In accordance with at least one embodiment of the present disclosure, the soil property sensor 312 may include an independent power source 350. The independent power source 350 may provide power to the systems of the soil property sensor 312. In some embodiments, the independent power source 350 facilitates independent operation of the soil property sensor 312. For example, the independent power source 350 may facilitate operation of the soil property sensor 312 without a wired connection to an irrigation connection. This may increase the ease of installation, maintenance, and operation of the soil property sensor 312. For example, wired connections in a landscape environment may be subject to damage, including damage from environmental factors (e.g., decay from ultraviolet radiation, moisture, wear, falling rocks), animals chewing on the wires, operators displacing, cutting, or otherwise damaging the wires during operation, any other damage, and combinations thereof.

Further, the wireless communication system 348 and independent power source 350 may improve the flexibility of placement of the soil property sensor 312. For example, the wireless communication system 348 and the independent power source 350 may allow the soil property sensor 312 to be placed at any location, and not locations that are impacted at least partially by wired power and/or communication lines. In this manner, the irrigation operator may place the soil property sensor 312 at any desired location, thereby increasing the accuracy and/or representation of the measurements by the soil property sensor 312.

In some embodiments, the interior of the soil property sensor 312 may be sealed. For example, the soil property sensor 312 may include a sensor cover 352. The sensor cover 352 may be secured to the housing 314 to form a waterproof seal to prevent the ingress of water to the interior of the housing 314. In some embodiments, the sensor cover 352 may be sized to cover an opening at the upper end of the housing. The opening may provide space for an operator to insert the sensors 338, the wireless communication system 348, the independent power source 350, any electronics or other elements, and combinations thereof, into the housing 314. The sensor cover 352 may be inserted into or onto the opening to form the waterproof seal. The joint between the sensor cover 352 and the opening of the housing 314 may be formed with a seal to prevent the ingress of water into the housing 314 through the opening. Forming a waterproof seal at the opening may protect the electronics and sensors from damage from the water.

The waterproof seal may be formed from any type of waterproof seal between the sensor cover and the housing 314. For example, the waterproof seal may include an O-ring. In some examples, the waterproof seal may include a caulk. In some examples, the waterproof seal may include an epoxy, weld, or braze. In some examples, the waterproof seal may include any seal that may prevent ingress of water into the interior of the housing 314.

In some embodiments, the elements in the interior of the housing 314 may be fully potted. For example, the sensors, electronics board, communication elements, power source, and other components in the interior of the soil property sensor 312 may be potted, or surrounded by an epoxy, resin, or other potting material. This may help to secure the components of the soil property sensor 312, reducing the potential for damage to the components, based on movement, dropping, jarring, hammering, or otherwise impacting the soil property sensor 312.

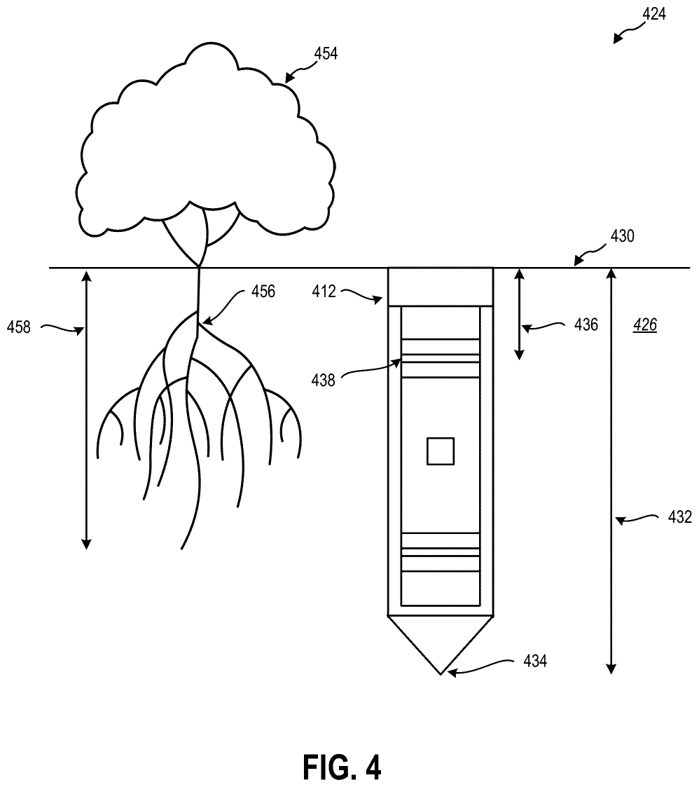

FIG. 4 is a representation of a soil monitoring system 424 with a soil property sensor 412 installed in the ground 426 adjacent to or proximate to a plant 454, according to at least one embodiment of the present disclosure. The plant 454 includes roots 456 that grow below the surface 430 of the ground 426. The roots 456 may absorb water and nutrients from the soil in the ground 426. The plant 454 may be any plant. For example, the plant 454 may include grass, trees, shrubs, flowers, bulbs, vegetables, fruits, berries, any other plant, and combinations thereof.

The soil property sensor 412 may be placed adjacent to or proximate to the plant 454. For example, the soil property sensor 412 may be placed within the root network or root flare of the plant 454. In some examples, the soil property sensor 412 may be placed underneath one or more leaves or branches of the plant 454. In some examples, the soil property sensor 412 may be placed within the same flower bed as the plant 454. In some examples, the soil property sensor 412 may be placed within the same soil type as the plant 454. In some examples, the soil property sensor 412 may be placed within the same field as the plant 454. In some examples, the soil property sensor 412 may be placed within the reach of the same irrigation device (e.g., sprinkler head, drip-system) as the plant 454. In some examples, the soil property sensor 412 may be placed within the same irrigation zone as the plant 454. Placing the soil property sensor 412 proximate the plant 454 may help to increase the representation of the measurements of the soil property sensor 412 to the conditions of the ground 426 at the plant 454.

Many plants absorb water and nutrients across an absorption zone 458. In some embodiments, the absorption zone 458 may be in a range having an upper value, a lower value, or upper and lower values including any of 0 in. (e.g., at least a portion of the roots 456 may be exposed at the surface 430), 0.5 in. (1.3 cm), 1 in. (2.5 cm), 1.5 in. (3.8 cm), 3 in. (7.6 cm), 6 in. (15.2 cm), 9 in. (22.9 cm), 12 in. (30.5 cm), 15 in. (38.1 cm), 18 in. (45.7 cm), 21 in. (53.3 cm), 24 in. (61.0 cm), or any value therebetween. For example, the absorption zone 458 may be greater than 0 in. In another example, the absorption zone 458 may be less than 24 in. (61.0 cm). In yet other examples, the absorption zone 458 may be any value in a range between 0 in. and 24 in. (61.0 cm).

In accordance with at least one embodiment of the present disclosure, the soil property sensor 412 may be at least partially installed in the absorption zone 458. This may allow the soil property sensor 412 to measure and/or monitor soil properties along the absorption zone 458. In some embodiments, the instrument length 432 of the soil property sensor 412 may be the same as the absorption zone 458. Put another way, the lower surface 434 of the soil property sensor 412 may be inserted below the surface 430 at a depth that is the same as or approximately the same as the absorption zone 458. In some embodiments, the lower surface 434 may be greater than or less than the absorption zone 458.

In some embodiments, the sensor depth 436 of one or more of the sensors 438 may be within the absorption zone 458. In this manner, any soil property measurements made by one or more of the sensors 438 may be representative of the conditions of the soil within the absorption zone 458.

As discussed herein, the soil property sensor 412 may include multiple sensors 438. Two different sensors that sense the same soil property may be located at different sensor depths 436 along the instrument length 432. Based on the measurements taken at different sensor depths 436, the soil monitoring system 424 may generate a soil property gradient. In some embodiments, the soil property gradient may allow the irrigation operator to determine the soil property along any portion of the absorption zone 458. In some embodiments, this may include inferring or determining the soil property between two sensors 438 along the instrument length 432.

In some embodiments, the sensor depth 436 may be greater than the absorption zone 458. This may allow the soil property gradient to encompass the entire portion of the absorption zone 458, or to encompass the bottom of the absorption zone 458. In this manner, the soil monitoring system 424 may identify the soil property at the bottom of the absorption zone 458, where moisture and/or nutrients may be less available.

In some embodiments, the sensor depth 436 may be less than the absorption zone 458. Using the soil property gradient, the soil monitoring system 424 may determine the soil property outside of the sensor depth 436. This may allow the soil monitoring system 424 to extrapolate the soil property. In some embodiments, the soil monitoring system 424 may extrapolate the soil property to the be within the absorption zone 458, including to the extends (both upper and lower) of the absorption zone 458.

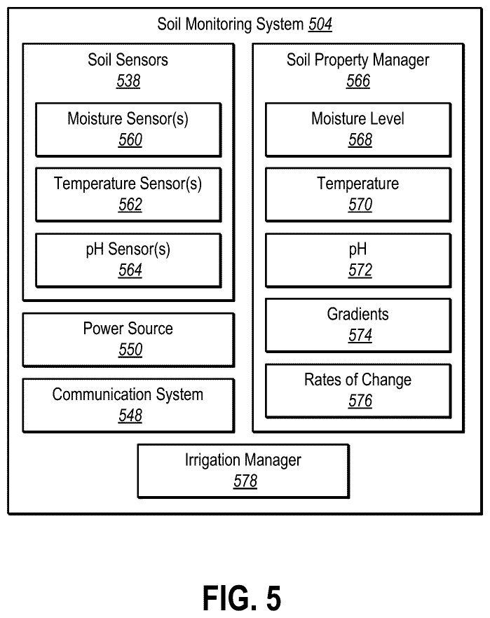

FIG. 5 is a representation of a soil monitoring system 524, according to at least one embodiment of the present disclosure. Each of the components of the soil monitoring system 524 can include software, hardware, or both. For example, the components can include one or more instructions stored on a computer-readable storage medium and executable by processors of one or more computing devices, such as a client device or server device. When executed by the one or more processors, the computer-executable instructions of the soil monitoring system 524 can cause the computing device(s) to perform the methods described herein. Alternatively, the components can include hardware, such as a special-purpose processing device to perform a certain function or group of functions. Alternatively, the components of the soil monitoring system 524 can include a combination of computer-executable instructions and hardware.

Furthermore, the components of the soil monitoring system 524 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The soil monitoring system 524 may monitor the soil properties at a soil property sensor (e.g., the soil property sensor 212 illustrated in FIG. 2). In some embodiments, the soil monitoring system 524 may analyze the soil properties and provide irrigation and/or landscaping recommendations to an irrigation controller or irrigation manager.

The soil monitoring system 524 may include a soil property sensor that includes one or more sensors 538. The sensors 538 may include any type of soil property sensor. For example, the sensors 538 may include moisture sensor(s) 560, temperature sensor(s) 562, soil chemistry sensors (such as pH sensor(s) 564), any other soil property sensor, and combinations thereof. In some embodiments, as discussed herein, the sensors 538 may be located along a length of the soil property sensor. In some embodiments, two of the same type of sensor 538 may be located at different lengths of the soil property sensor. The sensors 538 may collect multiple measurements of soil properties at different times, resulting in time-series soil property data.

The soil monitoring system 524 may include an independent power source 550. The independent power source 550 may provide power to the various components operating on the soil property sensor. In some embodiments, the independent power source 550 may provide power sufficient for the operating lifetime of the soil property sensor. In some embodiments, the independent power source 550 may be rechargeable. In some embodiments, the independent power source 550 may be rechargeable using a power generator located on the soil property sensor. For example, the soil property sensor may include a solar panel that may be used to charge the independent power source 550.

The soil monitoring system 524 may further include a wireless communication system 548. The wireless communication system 548 may communicate wirelessly with other elements of the irrigation system. In some embodiments, the wireless communication system 548 may include a low-power communication system, such as a LoRa communication system.

The soil monitoring system 524 may include a soil property manager 566. The soil property manager 566 may process and/or analyze the soil property measurements measured by the sensors 538. The soil property manager 566 may generate any type of analysis. For example, the soil property manager 566 may generate a moisture level 568 from measurements received from the moisture sensor(s) 560. The soil property manager 566 may generate a temperature 570 from measurements received from the temperature sensor(s) 562. The soil property manager 566 may generate a pH level from the pH sensor(s) 564.

The soil property manager 566 may associate a depth for each of the measurements and determined soil properties. For example, each of the sensors 538 may be located a sensor depth below the upper surface of the soil property sensor. When the soil property sensor is installed in the ground, the upper surface may be flush or approximately flush with the surface, and the sensor depth may be the same as the depth below the surface. In some embodiments, if the soil property sensor is not installed flush with the surface, the operator may identify how far above or below the surface the upper surface is installed, and modify the sensor depth to reflect the difference between the upper surface of the soil property sensor and the ground surface. Based on which sensor the soil property measurement was received from, the soil property manager 566 may associate the measurement depth with the sensor depth.

In some embodiments, the soil property manager 566 may determine one or more soil property gradients 574. The one or more soil property gradients 574 may be representations of the change in soil property along the depth of the soil property sensor. For example, the soil property manager 566 may receive two separate measurements of the same soil property from two or more separate sensors 538 located at different sensor depths. Based on the measured or determined soil properties, the soil property manager 566 may generate a soil property gradient 574.

In some embodiments, the soil property manager 566 may utilize the soil property gradient to determine soil properties at different depths than the sensor depths at which the soil properties were measured. For example, the soil property manager 566 may generate soil properties using the soil property gradient 574 at depths between the sensor depths at which the soil properties were measured. In some examples, the soil property manager 566 may generate soil properties using the soil property gradient 574 at depths less than or greater than any of the sensor depths at which the soil properties were measured. This may allow the operator to calculate or infer soil properties at any depth, thereby improving the relevance and/or representation of the soil properties to the actual conditions of the ground.

The soil property manager 566 may receive the time-series soil property data and generate rates of change for the soil properties. In some embodiments, the soil property manager 566 may generate rates of change 576 for the soil property gradients 574. This may help the soil property manager 566 to identify trends in the change of soil properties, such as the rate at which moisture is absorbed and/or drained, the rate at which the temperature changes across the depth of the soil, the rate at which the pH changes across the depth of the soil, any other rate of change, and combinations thereof.

The soil monitoring system 524 may further include an irrigation manager 578. The irrigation manager 578 may receive the soil properties, including the soil property gradients 574 and/or the rates of change 576 of the soil properties. For example, the irrigation manager 578 may identify that the moisture content at a depth below the soil is outside of a threshold or threshold range. The irrigation manager 578 may provide an irrigation recommendation to change the watering duration and/or frequency to return the moisture content to within the threshold or threshold range. In some examples, the irrigation manager 578 may identify that the moisture content of the soil decreases to below a minimum threshold between waterings. Based on this rate of change of the moisture gradient, the irrigation manager 578 may prepare a recommendation to change the watering duration and/or frequency. In some examples, the irrigation manager may prepare landscaping recommendations, such as a change in plant type to match the water conditions, including drought-tolerant plants for low-water conditions, moisture-loving plants for high-water conditions, and so forth. In some examples, the irrigation manager may prepare landscaping and/or irrigation recommendations based on any other property, such as watering times based on temperature, fertilizer and/or plant types based on pH, fertilizer frequency based on the rate of change of pH, any other landscaping and/or irrigation recommendation, and combinations thereof.

In accordance with at least one embodiment of the present disclosure, the soil monitoring system 524 may perform at least a portion of the determinations of the soil properties and/or irrigation recommendations at the soil property sensor. This may help to reduce the amount of information communicated by the wireless communication system 548, thereby reducing the power consumption of the wireless communication system 548. In some embodiments, the soil property sensor may transmit the raw measurements and/or partially processed measurements to an irrigation controller, and the irrigation controller may at least partially determine the soil properties and/or recommendations.

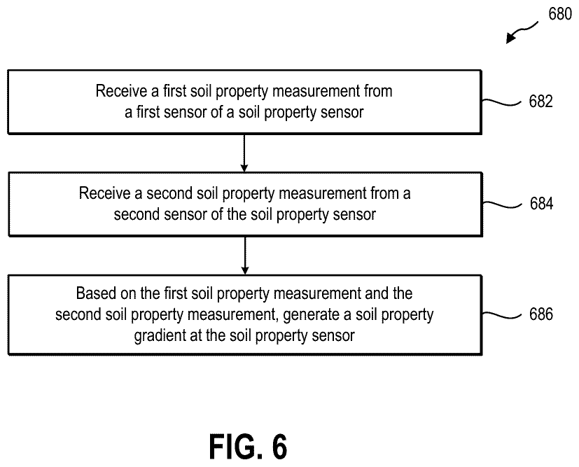

FIG. 6, the corresponding text, and the examples provide a number of different methods, systems, devices, and computer-readable media of the soil monitoring system. In addition to the foregoing, one or more embodiments can also be described in terms of flowcharts comprising acts for accomplishing a particular result, as shown in FIG. 6. FIG. 6 may be performed with more or fewer acts. Further, the acts may be performed in differing orders. Additionally, the acts described herein may be repeated or performed in parallel with one another or parallel with different instances of the same or similar acts.

As mentioned, FIG. 6 illustrates a flowchart of a series of acts or a method 680 for irrigation management, according to at least one embodiment of the present disclosure. While FIG. 6 illustrates acts according to one embodiment, alternative embodiments may omit, add to, reorder, and/or modify any of the acts shown in FIG. 6. The acts of FIG. 6 can be performed as part of a method. Alternatively, a computer-readable medium can comprise instructions that, when executed by one or more processors, cause a computing device to perform the acts of FIG. 6. In some embodiments, a system can perform the acts of FIG. 6.

A soil monitoring system may receive a first soil property measurement from a first sensor of a soil property sensor at 682. The first sensor may be located a first sensor depth below the surface. The soil monitoring system may receive a second soil property measurement from a second sensor of the soil property sensor at 684. The second sensor may be located at a second depth below the surface. The soil monitoring system may, based on the first soil property measurement and the second soil property measurement, generate a soil property gradient at the soil property sensor at 686.

In accordance with at least one embodiment of the present disclosure, the soil monitoring system, based on the soil property gradient, adjusts a watering program of the irrigation system. In some embodiments, the first soil property measurement includes a plurality of first soil property measurements and the second soil property measurement includes a plurality of second soil property measurements. The soil monitoring system may generate a rate of change of the soil property gradient based on the plurality of first soil property measurements and the plurality of second soil property measurements. In some embodiments, the soil monitoring system adjusts a watering program of the irrigation system based on the rate of change of the soil property gradient.

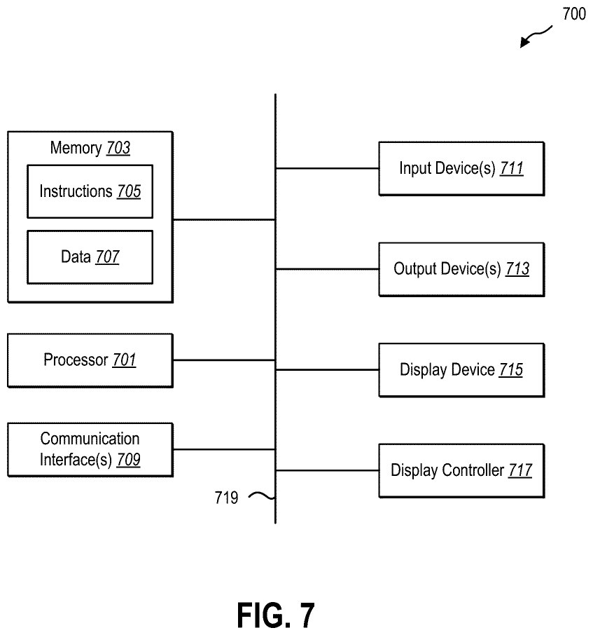

FIG. 7 illustrates certain components that may be included within a computer system 700. One or more computer systems 700 may be used to implement the various devices, components, and systems described herein.

The computer system 700 includes a processor 701. The processor 701 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 701 may be referred to as a central processing unit (CPU). Although just a single processor 701 is shown in the computer system 700 of FIG. 7, in an alternative configuration, a combination of processors (e.g., an ARM and DSP) could be used.

The computer system 700 also includes memory 703 in electronic communication with the processor 701. The memory 703 may be any electronic component capable of storing electronic information. For example, the memory 703 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 705 and data 707 may be stored in the memory 703. The instructions 705 may be executable by the processor 701 to implement some or all of the functionality disclosed herein. Executing the instructions 705 may involve the use of the data 707 that is stored in the memory 703. Any of the various examples of modules and components described herein may be implemented, partially or wholly, as instructions 705 stored in memory 703 and executed by the processor 701. Any of the various examples of data described herein may be among the data 707 that is stored in memory 703 and used during execution of the instructions 705 by the processor 701.

A computer system 700 may also include one or more communication interfaces 709 for communicating with other electronic devices. The communication interface(s) 709 may be based on wired communication technology, wireless communication technology, or both. Some examples of communication interfaces 709 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 700 may also include one or more input devices 711 and one or more output devices 713. Some examples of input devices 711 include a keyboard, mouse, microphone, remote control device, button, joystick, trackball, touchpad, and lightpen. Some examples of output devices 713 include a speaker and a printer. One specific type of output device that is typically included in a computer system 700 is a display device 715. Display devices 715 used with embodiments disclosed herein may utilize any suitable image projection technology, such as liquid crystal display (LCD), light-emitting diode (LED), gas plasma, electroluminescence, or the like. A display controller 717 may also be provided, for converting data 707 stored in the memory 703 into text, graphics, and/or moving images (as appropriate) shown on the display device 715.

The various components of the computer system 700 may be coupled together by one or more buses, which may include a power bus, a control signal bus, a status signal bus, a data bus, etc. For the sake of clarity, the various buses are illustrated in FIG. 7 as a bus system 719.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A soil property sensor, comprising:
a housing having an opening;
a plurality of sensors located in the housing, the plurality of sensors including:
a first soil property sensor located at a first sensor depth on the housing; and
a second soil property sensor located at a second sensor depth on the housing;
a power source located in the housing, the power source providing power to the plurality of sensors;
a wireless communication system in the housing; and
a sensor cover at the opening, the sensor cover forming a waterproof seal into the opening.

2. The soil property sensor of claim 1, wherein the plurality of sensors includes a pH sensor.

3. The soil property sensor of claim 2, wherein the plurality of sensors includes a temperature sensor.

4. The soil property sensor of claim 1, wherein the plurality of sensors, the power source, and the wireless communication system are potted within the housing.

5. The soil property sensor of claim 1, wherein the housing includes a plurality of retention features.

6. The soil property sensor of claim 1, further comprising a processor and memory, the memory including instructions that cause the processor to determine a soil property gradient based on the first soil property sensor and the second soil property sensor.

7. A method for soil monitoring in an irrigation system, the method comprising:
receiving a first soil property measurement from a first sensor of a soil property sensor inserted in soil, the first sensor located at a first sensor depth;
receiving a second soil property measurement from a second sensor of the soil property sensor, the second sensor located at a second sensor depth; and
based on the first soil property measurement and the second soil property measurement, generating a soil property gradient of said soil at the soil property sensor.

8. The method of claim 7, further comprising, based on the soil property gradient, adjusting a watering program of the irrigation system.

9. The method of claim 8, wherein adjusting the watering program includes adjusting at least one of a watering duration or watering schedule.

10. The method of claim 7, wherein the first soil property measurement includes a plurality of first soil property measurements and the second soil property measurement includes a plurality of second soil property measurements, and further comprising generating a rate of change of the soil property gradient based on the plurality of first soil property measurements and the plurality of second soil property measurements.

11. The method of claim 10, further comprising, based on the rate of change of the soil property gradient, adjusting a watering program of the irrigation system.

12. The method of claim 7, wherein receiving the first soil property measurement includes receiving a first moisture measurement and receiving the second soil property measurement includes receiving a second moisture measurement.

13. The method of claim 7, further comprising, using the soil property gradient, generating a soil property at a depth that is different than the first sensor depth or the second sensor depth.

14. A soil monitoring system, comprising:
a soil property sensor, including:
a first sensor located a first sensor depth on the soil property sensor; and
a second sensor located a second sensor depth on the soil property sensor; and
a processor and memory, the memory including instructions that cause the processor to:
measure a first soil property measurement with the first sensor of a soil property sensor inserted in soil;
measure a second soil property measurement with the second sensor of the soil property sensor; and
based on the first soil property measurement and the second soil property measurement, generating a soil property gradient of said soil at the soil property sensor.

15. The soil monitoring system of claim 14, wherein the instructions further cause the processor to, based on the soil property gradient, adjusting a watering program of the soil monitoring system.

16. The soil monitoring system of claim 15, wherein adjusting the watering program includes adjusting at least one of a watering duration or watering schedule.

17. The soil monitoring system of claim 14, wherein the first soil property measurement includes a plurality of first soil property measurements and the second soil property measurement includes a plurality of second soil property measurements, and wherein the instructions further cause the processor to generate a rate of change of the soil property gradient based on the plurality of first soil property measurements and the plurality of second soil property measurements.

18. The soil monitoring system of claim 17, wherein the instructions further cause the processor to, based on the rate of change of the soil property gradient, adjust a watering program of the soil monitoring system.

19. The soil monitoring system of claim 14, wherein measuring the first soil property measurement includes measuring a first moisture measurement and measuring the second soil property measurement includes measuring a second moisture measurement.

20. The soil monitoring system of claim 14, wherein the instructions further cause the processor to, using the soil property gradient, generate a soil property at a depth that is different than the first sensor depth or the second sensor depth.
